(19) United States
(12) Patent Application Publication
(10) Pub. No.: US 2012/0253323 A1
(43) Pub. Date: Oct. 4, 2012

(54) ROTATIONALLY DRIVEN SURGICAL TOOL ASSEMBLY AND METHOD

(75) Inventors: Jeetendra Bharadwaj, Memphis, TN (US); Bradley Steele, Germantown, TN (US); Thoams McGahan, Memphis, TN (US); Jacob Zimmerman, North Little Rock, AR (US)

(73) Assignee: WARSAW ORTHOPEDIC, INC., Warsaw, IN (US)

(21) Appl. No.: 13/074,875

(22) Filed: Mar. 29, 2011

(51) Int. Cl. A61B 17/00

(52) U.S. Cl. 606/1

(57) ABSTRACT

A surgical tool assembly typically includes a drive unit and a surgical tool. The drive unit includes a female drive recess for receiving the proximal end of the surgical tool. A guard is disposed in front of the drive recess and acts as a filter to limit the possible surgical tools that may be mated with the drive unit. The guard includes a non-circular filter opening with a minimum cross dimension that is larger than a minimum cross dimension of the drive recess, but smaller than the maximum cross dimension of the drive recess. Proper surgical tools include a non-round keying section that is configured to fit through the guard opening in at least one orientation. The guard is spaced sufficiently forward relative to the drive recess so that only properly keyed surgical tools may be mated with the drive recess.

### BACKGROUND

The present invention relates to rotationally driven surgical instruments and methods of using the same, and more particularly to rotationally driven surgical instruments that are releasably coupled to a rotational drive unit.

During surgery, particularly spinal surgeries, it is common to use surgical tools that are rotationally driven. For example, a surgical tool may be rotationally driven in order to spin a surgical implement for cutting bone or the like. Typically, such tools include an elongate shaft that has the surgical implement on the distal end and a drive section on the proximal end. The proximal drive section is releasably mated to a corresponding drive section in the drive unit. When coupled together and activated, the drive unit causes the surgical tool to rotate to achieve the desired effect. Typically, the drive sections take the form of conventional square drive connections, with the surgical tool being the male portion, and the drive section in the drive unit being the female portion. If desired, there may be suitable biased ball detents or other known means for allowing the connection to be a reversible snap-fit connection.

While such arrangements have found wide acceptance, they have not proven desirable for all situations. For example, it may be desired to limit the array of possible surgical tools that can be mated to a given drive unit. However, if all the available surgical tools have the same size square drives, then there is little or nothing to prevent each of those tools from being operably mated to the drive unit, even if some are inappropriate for use with that particular drive unit. As such, there remains a need for alternative approaches to surgical tools and drive units.

### SUMMARY

The present invention provides for a surgical tool assembly that typically includes a drive unit and a surgical tool. The drive unit includes a female drive recess for receiving the proximal end of the surgical tool. A guard is disposed in front of the drive recess and acts as a filter to limit the possible surgical tools that may be mated with the drive unit. Thus, the guard acts as a mechanical filter to limit access to the drive recess, allowing surgical tools with proper configurations to be joined to the drive unit, but not other surgical tools with improper configurations.

According to one aspect, the present invention provides a surgical tool assembly comprising a driving unit. The driving unit comprises a rotational power source, an outer sleeve, a female driving section, and a filter. The female driving section is coupled to the outer sleeve for rotation about a first rotational axis when receiving rotational input from the rotational power source. The female drive section has a non-circular drive recess therein, with the drive recess opening distally and having a maximum cross-sectional dimension, normal to the first axis, of X. The drive recess also has a minimum cross-sectional dimension, normal to the first axis, of Z. The filter is mounted to the outer sleeve distally from the drive recess and freely rotatable relative to the outer sleeve. The filter has a filter opening therethrough with a first non-circular cross-section. The filter opening has a minimum cross-sectional dimension, normal to the first axis, of Y. Dimension Y is less than dimension X and more than dimension Z. The drive recess and the filter opening are aligned along the first axis and separated by a first distance. The surgical tool assembly may further comprise a surgical tool removably coupled to the driving unit. The surgical tool may comprise an elongate drive shaft, a male driven section, a keying feature; a distal working end. The elongate drive shaft extends along a longitudinal axis from a proximal section to a distal section. The male driven section is disposed at a proximal end of the drive shaft. The male driven section has a non-circular cross-section normal to the longitudinal axis and a proximal endface. The male driven section has a maximum cross-sectional dimension, normal to the longitudinal axis, of approximately X. The male driven section has a minimum cross-sectional dimension, normal to the longitudinal axis, of approximately Z. The keying feature is spaced from the distal endface by a second distance shorter than the first distance. The keying feature having a non-circular cross-section. The keying feature is configured to fit through the filter opening of the filter. The female driving section and the male driven section are mated together to rotationally couple the surgical tool to the female driving section. The filter is rotationally coupled to the female driving section only through the surgical tool. A distance D between a proximal edge of the keying feature and the proximal endface is shorter than the first distance.

In another aspect, the present invention provides a surgical tool assembly comprising a driving unit and a surgical tool removably coupleable to the driving unit. The surgical tool comprises an elongate drive shaft, a male driven section, an interference feature, and a distal working end. The elongate drive shaft extends along a first rotational axis. The male driven section is disposed at a proximal end of the drive shaft. The male driven section has a first non-circular cross-sectional profile normal to the first axis and a proximal endface. The interference feature is disposed distally from the proximal endface of the drive section by a first distance. The interference feature has a first dimension in a radial direction larger than a corresponding portion of the cross-sectional profile of the male drive section. The driving unit comprises a rotational power source, a female driving section, an outer sleeve, and a guard. The female driving section is supported for rotation about a first axis. The female driving section has a distally open drive recess configured to receive the male driven section. The outer sleeve extends distally from the female driving section and is rotationally coupled to the female driving section. The guard is mounted to the outer sleeve distally from the drive recess. The guard has a gate opening therethrough with a second cross-sectional profile that is non-circular, with the first and second cross-sectional profiles being different. The drive recess and the gate opening are aligned along the first axis and separated by a second distance larger than the first distance. A theoretical cylinder with a diameter of the first dimension cannot pass into the gate opening of the guard. The assembly is reversibly convertible between a first configuration and a second configuration. In the first configuration: 1) the surgical tool is decoupled from the driving unit; and 2) the guard is freely rotatable relative to the outer sleeve for rotation about the first axis. In the second configuration: 1) the male driven section is matingly received in the drive recess to rotationally couple the surgical tool to the female driving section; 2) the interference feature extends into the gate opening of the guard; 3) the working end of the surgical tool is disposed distally from the outer sleeve in spaced relation thereto; and 4) the guard is rotationally coupled to the female driving section through the surgical tool. The rotational power source, female driving section, guard, and surgical tool are configured such that, in the second configuration, rotational power from the rotational power source is supplied to the surgical tool via the female driving section.

In another aspect, the present invention provides a surgical tool assembly comprising: a driving unit and a surgical tool removably coupleable to the driving unit. The driving unit comprises a housing; a female driving section, and a filter. The female driving section is rotatably coupled to the housing. The female driving section has a drive recess therein having the first cross-sectional profile. The filter is mounted to the housing distally from the recess. The filter has a keyed gate opening therethrough with a second cross-sectional profile that is non-circular. The first and second cross-sectional profiles are different. The drive recess and the gate opening are aligned along a first axis and separated by a first distance. The drive recess rotates about the first axis when the female driving section is driven. The surgical tool comprises an elongate drive shaft and a distal working end. The elongate drive shaft extends along a longitudinal axis from a proximal section to a distal section. The proximal section includes a male driven section disposed at a proximal end of the drive shaft and an interference feature disposed distally therefrom. The male driven section has a third non-circular cross-sectional profile normal to the longitudinal axis and a proximal endface. The interference feature having a fourth non-circular cross-sectional profile disposed distally from the proximal endface of the drive section by a second distance, with the second distance shorter than the first distance. The interference feature has at least a first dimension in a radial direction larger than any radial dimension of the male driven section. The interference feature sized and configured to fit through the keyed gate opening of the filter. With the male driven section mated to the female driving section: 1) the second cross-section entirely envelopes the first cross-section when viewed along the longitudinal axis; 2) the surgical tool rotationally couples the female driving section to the filter; and 3) a most-proximal portion of the interference feature is disposed between the filter and the female driving section.

In various embodiments, the present invention has one or more of the above attributes, alone or in any combination.

### BRIEF DESCRIPTION OF THE DRAWINGS

FIG. 1 shows a surgical tool assembly according to one or more embodiments in an assembled condition.

FIG. 2 shows a drive section of one embodiment in perspective view.

FIG. 3 shows a front view of the drive section of FIG. 2

FIG. 4 shows a front view of a guard showing the gate or filter opening, according to one embodiment.

FIG. 5 shows a perspective view of the surgical tool of FIG. 1.

FIG. 6 shows a detail view of the proximal section of the surgical tool of FIG. 5.

FIG. 7 shows a cross-section of the surgical tool of FIG. 5 along the line VII-VII of FIG. 5.

FIG. 8 shows a quick-connect mechanism that may be used with one or more embodiments.

FIG. 9 shows an exploded view of the quick-connect mechanism of FIG. 8.

FIG. 10 shows a cross-section of the quick-connect mechanism of FIG. 8 in the locked position, with the surgical tool omitted for clarity.

FIG. 11 shows an alternative surgical tool suitable for use with the guard of FIG. 4.

FIG. 12 shows an alternative guard with protrusions narrowing the gate opening and a corresponding surgical tool with longitudinal grooves.

### DESCRIPTION OF THE ILLUSTRATED EMBODIMENTS

The present invention provides for a surgical tool assembly that typically includes a drive unit and a surgical tool. The drive unit includes a female drive recess for receiving the proximal end of the surgical tool. A guard is disposed in front of the drive recess and acts as a filter to limit the possible surgical tools that may be mated with the drive unit. Thus, the guard acts as a mechanical filter to limit access to the drive recess. The guard includes a guard or filter opening that has a cross-sectional profile that is non-circular, with a minimum cross dimension that is larger than a minimum cross dimension of the drive recess, but smaller than the maximum cross dimension of the drive recess. Thus, a theoretical cylinder that has a diameter equal to the maximum cross dimension of the drive recess will not fit through the filter. Accordingly, the proper surgical tools include a non-round keying section disposed distally relative to the proximal end of the surgical tool that is configured to fit through the guard opening in at least one orientation. The guard is spaced sufficiently forward relative to the drive recess so that only properly keyed surgical tools may be mated with the drive recess. Additional details and embodiments are discussed below.

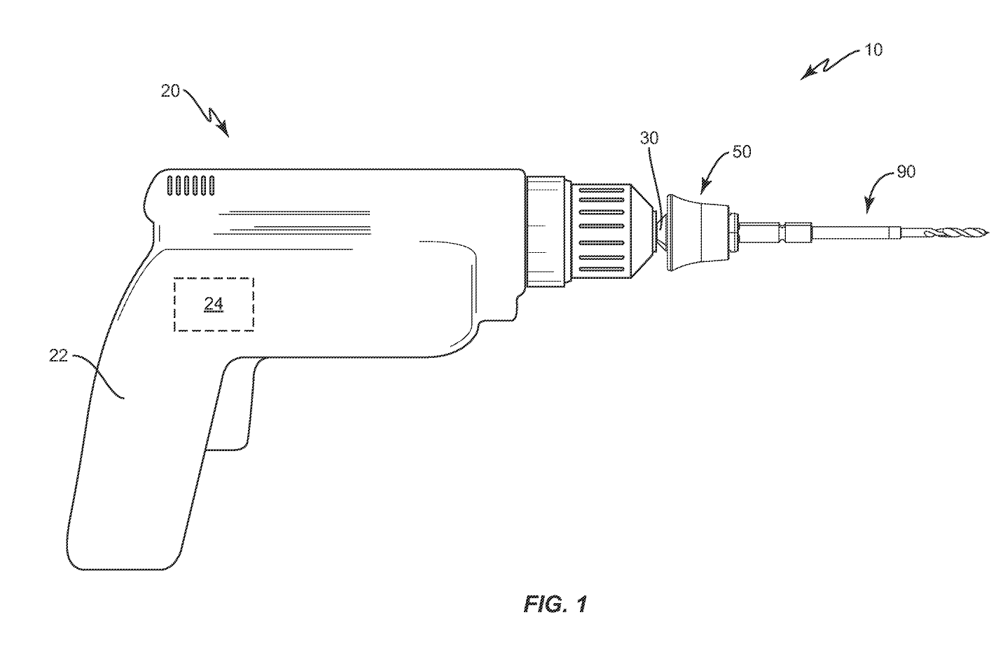

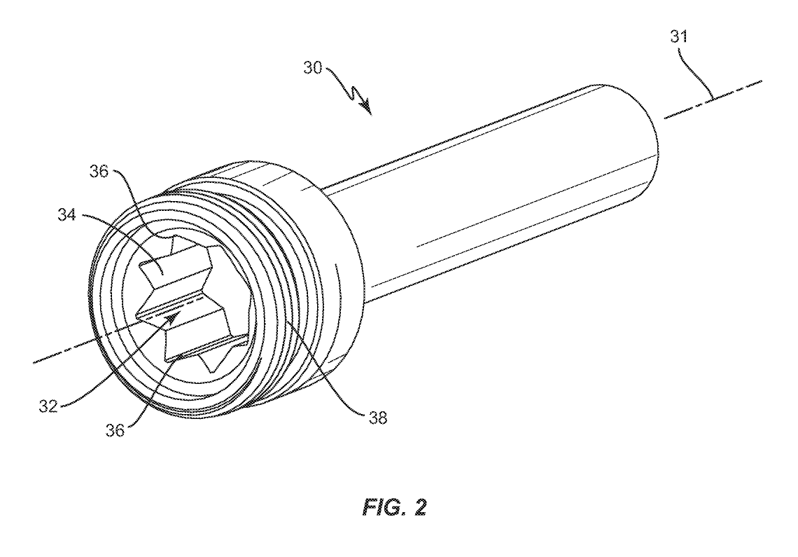

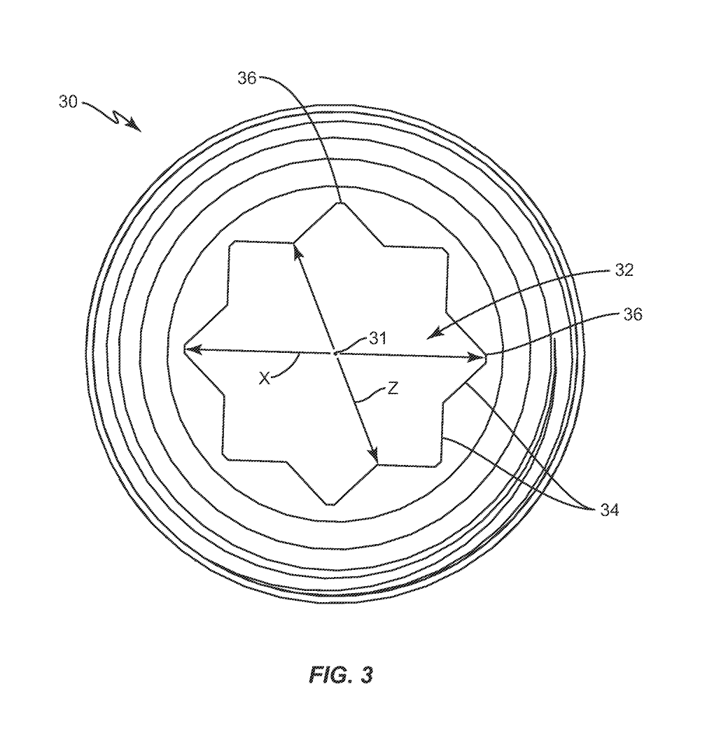

FIG. 1 shows an exemplary surgical tool assembly, generally designated 10, in an assembled state. The surgical tool assembly 10 includes a driving unit 20 and a surgical tool 90 releasably mated thereto. The driving unit 20 includes a housing 22, a female driving section 30, and a mechanical filter or guard 40. The housing 22 provides a convenient means of gripping the driving unit 20, and may take any form known in the art. A rotational drive 24 is advantageously disposed in or on the housing 22. The rotational drive may be an electric motor, a pneumatic motor, a manual crank with or without a gear train, or any other suitable means for supplying a rotational force on demand. The female driving section 30 is mounted to the housing 22 and operatively connected to the rotational drive 24 for rotation about a rotational axis 31. Referring to FIGS. 2-3, the distal end of the female driving section 30 includes a distally oriented drive recess 32 for mating with the surgical tool 90, as discussed further below. The drive recess 32 has a non-circular cross section (normal to axis 31) so that rotational force input to the female driving section 30 is transmitted to the surgical tool 90 when the tool 90 is mated to the female driving section 30. As such, the cross section of drive recess 32 may be any suitable shape, including square drive, a double square drive, hexalobular drive, star, triangular, oval, or any other suitable symmetric or asymmetric non-round shape. For purposes of illustration, it will be assumed that the drive recess 32 has a double square cross section. A double square cross section is conceptually two superimposed square cross-sections, with one rotated 45° relative to the others. The result is a cross-section profile with eight corners rather than four, see FIG. 3. As shown in FIG. 3, the drive recess 32 therefore has a minimum cross-sectional dimension of Z, and a maximum cross-sectional dimension of X. As used herein, the term “minimum cross-sectional dimension” means the smallest cross-sectional dimension that extends through the corresponding axis of rotation at a longitudinal midpoint of the relevant feature. Likewise, the term “maximum cross-sectional dimension” means the largest cross-sectional dimension that extends through the corresponding axis of rotation at a longitudinal midpoint of the relevant feature. Thus, for the square drive recess 32 shown in FIG. 3, the minimum cross-sectional dimension Z extends between opposing flats 34, while the maximum cross-sectional dimension X extends between opposing corners 36 of the illustrated square drive.

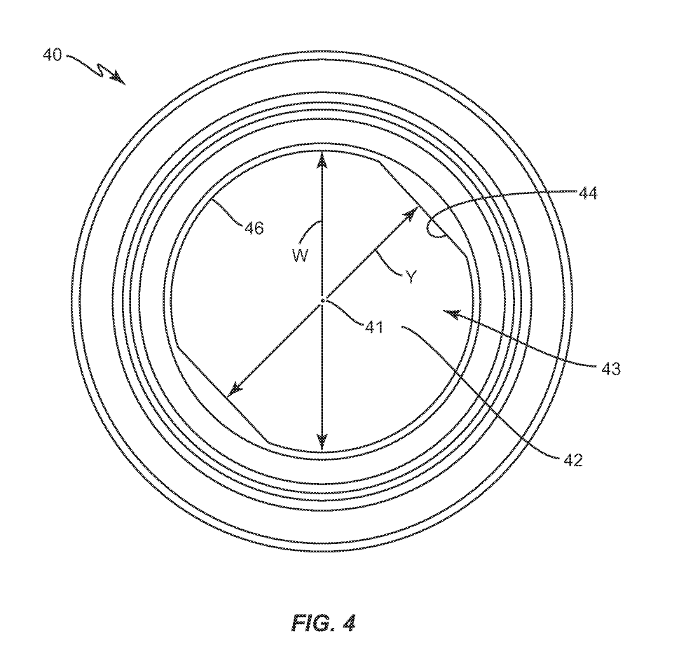

The guard 40 provides a limitation on access to the female driving section 30. The guard 40 is supported for free rotation about axis 41 and includes a central opening 42 sometimes referred to herein as the gate opening or a filter opening. See FIG. 4. The gate opening 42 is aligned with the female drive recess 32 so that axis 41 is collinear with axis 31. The gate opening 42 is non-circular in cross section, with a different cross-sectional profile than the drive recess 32. One exemplary profile is shown in FIG. 4. As can be seen, the profile includes a pair of opposed flats 44 separated by curving section sections 46. The curving sections are advantageously sections of a circle centered on axis 41. Thus, the flats 44 effectively narrow the gate opening 42 from the diameter of the circle to a smaller value. For such a configuration, the minimum cross-sectional dimension is between the flats 44 and represented at Y, and the maximum cross-sectional dimension is between the curving sections 46 and represented by W. The gate opening 42 is disposed distally (forwardly) relative to the female driving section 30, with the distance therebetween being C.

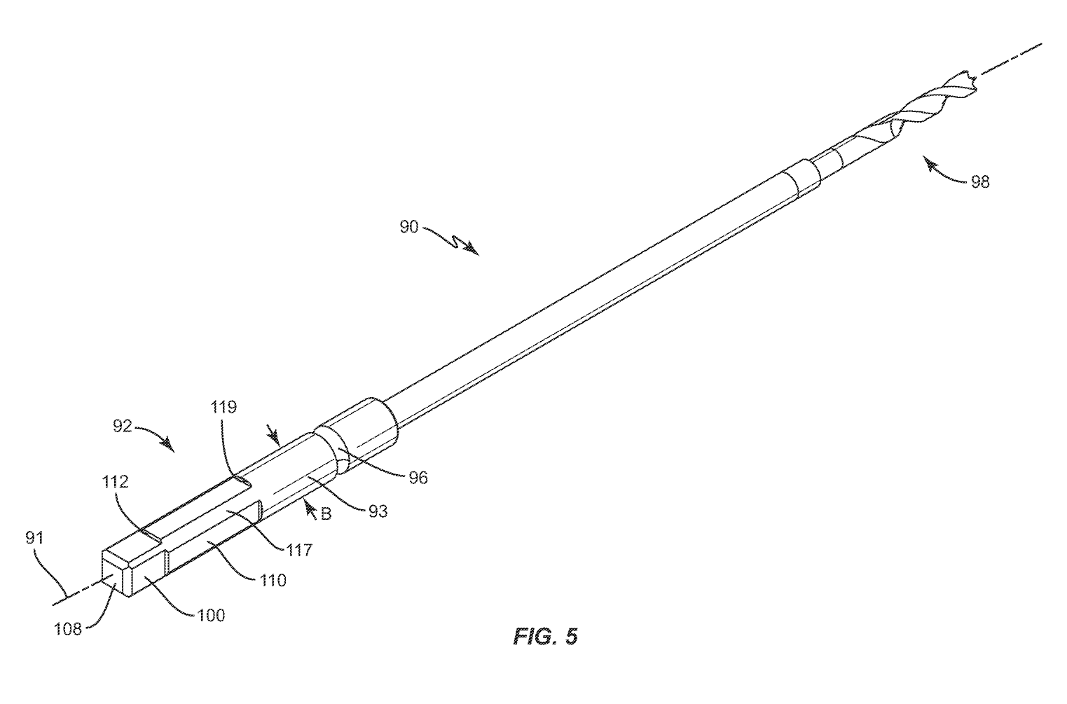

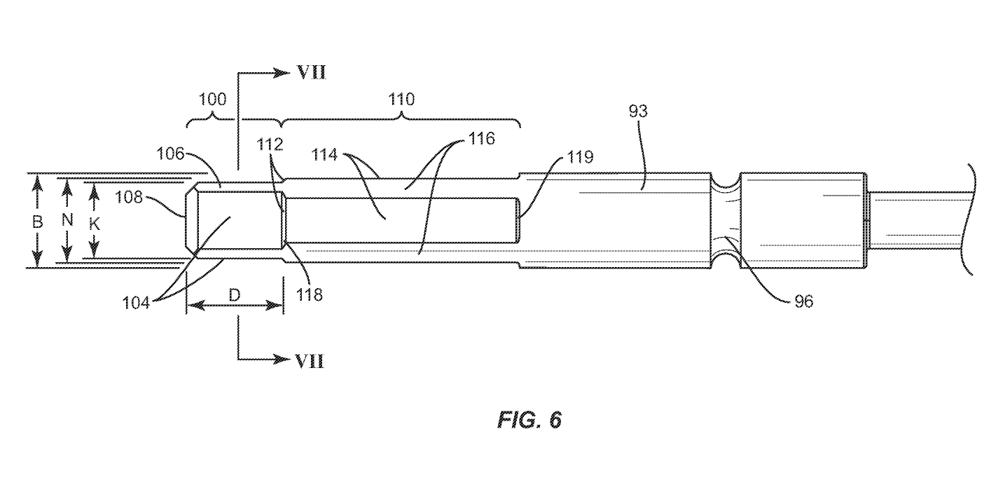

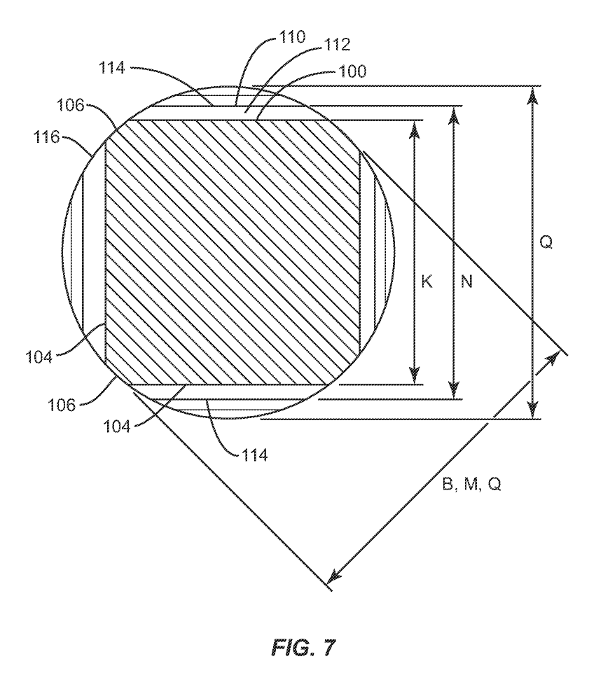

The surgical tool 90 is releasably attached to the female driving section 30 for rotation therewith. The surgical tool 90 extends along a longitudinal axis 91 from a proximal section 92 to a distal working end 98. See FIGS. 5-7. The working end 98 may be of any desired type, such as a bone drill bit, screwdriver, tap, etc., that works by rotating. The proximal section 92 includes a base section 93, a male driven section 100, and a keying section 110. The base section 93 is disposed closer to the working end 98 than the male driven section 100, with the keying section 110 disposed between the base section 93 and the male driven section 100. The base section 93 is generally cylindrical, with a diameter B.

The male driven section 100 is intended to engagingly mate with drive recess 32 of female driving section 30. As such, the male driven section 100 has a cross section suitable for mating with the drive recess 32 of female driving section 30. This cross section may be the same shape as drive recess 32 or may be slightly different. For the embodiment of FIGS. 5-7, the male driven section 100 has a square drive with flats 104 and corners 106. The flats 104 are spaced from each other by distance K, which represents the minimum cross-sectional dimension for the male driven section 100. The corners 106 are disposed between the flats, and are spaced from each other across axis 91 by distance M, which represents the maximum cross-sectional dimension of the male driven section. It should be noted that dimensions K and M of the made drive section 100 are less than dimensions Y and W of the gate opening 42, so that the male driven section 100 easily pass through the gate opening 42. Also, distance M may be the same as diameter B so that the corners 106 are partial extensions of the cylindrical exterior surface of base section 93. The male driven section 100 terminates in a proximal endface 108.

The keying section 110 is disposed between the male driven section 100 and the base section 93, advantageously with shoulders formed with its juncture of each. The keying section 110 has a cross-sectional profile that allows keying section 110 to pass through gate opening 42 in guard 40. Thus, the keying section 110 of FIGS. 5-7 has opposing flats 114 separated by adjoining curving sections 116. For such a configuration, the minimum cross-sectional dimension is between the flats 114 and represented by N, and the maximum cross-sectional dimension is between the curving sections 116 and represented by Q. In order that keying section 110 can fit into gate opening 42, dimensions N and Q are approximately the same, but slightly less than, dimensions Y and W of the gate opening 42. In some embodiments, the keying section 110 may have two flats 114. However, the keying section 110 advantageously has four flats 110 symmetrically disposed about axis 91 so that the keying section 110 may pass through gate opening 42 in any one of four relative rotational orientations. Also, distance Q may be the same as diameter B so that the corners 166 are partial extensions of the cylindrical exterior surface of base section 93. This allows the corners 116 to form longitudinally running bearing surfaces 117 for aiding in longitudinally locking the surgical tool relative to driving unit 20, as discussed further below. Because the keying section 110 is larger than the male driven section 100 in at least one dimension normal to axis 91, the proximal edge 118 of keying section 110 advantageously forms a proximally facing shoulder 112 at their juncture. This shoulder 112 is spaced forwardly from endface 108 by distance D Likewise, because keying section 110 is advantageously smaller than the base section 94 in at least one dimension normal to axis 91, the distal edge 119 of keying section 110 advantageously forms a proximally facing shoulder 119a at the junction with base section 93.

The surgical tool 90 is mated to the drive unit 20 by inserting the proximal section 92 of the surgical tool 90 into the gate opening 42 of the guard 40. As pointed out above, the male driven section 100 should pass through the gate opening 42 without difficulty. When the surgical tool 90 is inserted far enough that the proximal edge 118 of the keying section 110 meets the distal face of the guard 40, the surgical tool 90 should be rotationally oriented so that the keying section 110 can fit into the gate opening 42. For the exemplary embodiment, the two of flats 114 of the keying section 110 should be aligned with the flats 44 of the gate opening 42, with the curving sections 116 of the keying section 110 aligned with the curving sections 46 of the gate opening 42. Because the dimension N is slightly less than dimension Y for the flats and dimension Q is slightly less than dimension W for the curving sections, the keying section 110 should be able to pass into the gate opening 42 without being blocked when properly aligned. As can be appreciated, having cross-sectional profiles that are symmetric allows for more than one relative alignment between the keying section 110 and the gate opening 42 to be proper. For the exemplary embodiment of FIGS. 5-7, there are four relative orientations, 90° apart, that are acceptable. For other embodiments, there may be one, two, three, or more relative orientations that are acceptable.

If the guard 40 blocks the keying section 110 from entering the gate opening 42, then the male driven section 100 cannot be seated in the drive recess 32, and therefore cannot be properly engaged with the female driving section 30. This results from the distance C being larger than distance D, so the male driven section 100 cannot be seated in the drive recess 32 because the shoulder 112 of keying section 110 cannot pass into gate opening 42. Without proper engagement of the male driven section 100 in the drive recess 32, the rotation of the female driving section 30 will not result in rotation of the surgical tool 90. However, if the size and shape of the keying section 110 is correct, then the keying section 110 can pass through gate opening 42, and the male driven section 100 can be properly seated in the drive recess 32. Note that the entire keying section 110 need not pass through the gate opening 42, provided the proximal edge 118 passes thereinto or therethrough sufficiently to allow endface 108 of surgical tool 90 to be seated in drive recess 32 so that the flats 34, 104 (or other similar engagement features) can inter-engage. The surgical tool 90 is then longitudinally fixed relative to the drive unit 20 by any known means, such as using set screws, ring/hose clamps, ball-and-detent mechanisms, or the like. See also the discussion of a quick-connect mechanism below. The rotational drive 24 is then activated to rotate female driving section 30, which in turn causes the surgical tool 90 to rotate, allowing the working end 98 to be rotated to be used as desired by the surgeon. Note also that, depending on the relative position and length of the keying section 110, the rotation of the surgical tool 90 causes the guard 40 to rotate when the surgical tool 90 is rotated.

If it is desired to change to a different surgical tool 90, one having perhaps a different type working end 98 but the same proximal section 92 configuration, the surgeon need only pull the surgical tool 90 out of the drive unit 20 and insert the new one.

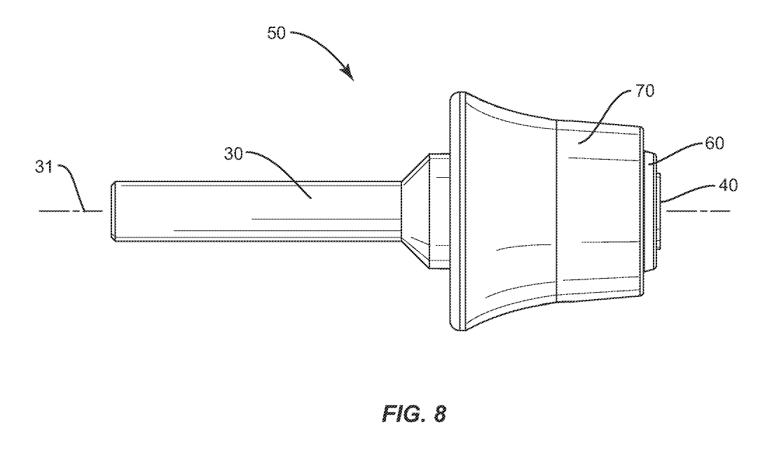

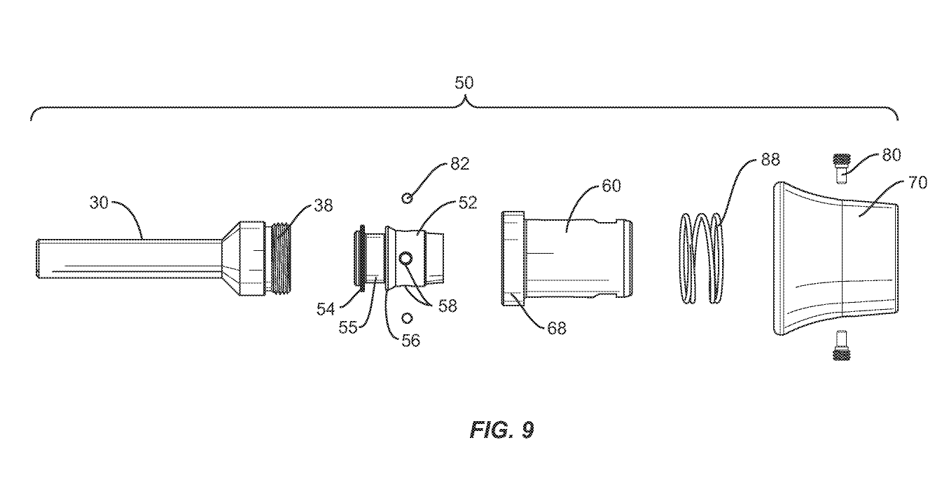

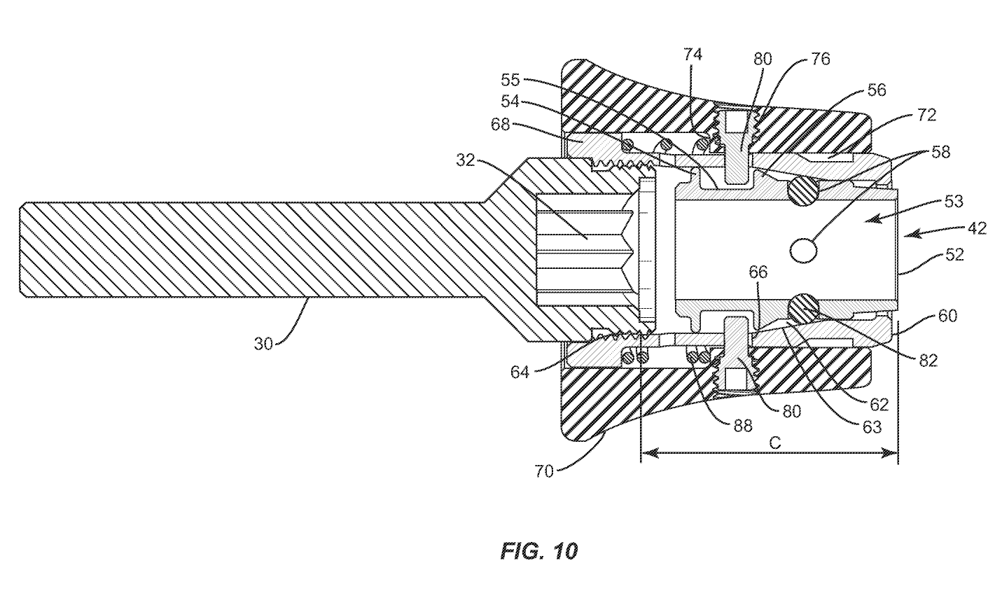

In some embodiments, a quick-connect assembly 50 may be used to facilitate the releasable longitudinal locking of the surgical tool 90 relative to the drive unit 20. Referring to FIGS. 8-10, an exemplary quick-connect assembly 50 is shown. The quick-connect assembly 50 includes an inner sleeve 52, an intermediate sleeve 60, an outer sleeve 70, retaining balls 82, and a spring 88. The inner sleeve 52 is generally tubular and includes the guard 40 on its distal end, a distal collar flange 56 located approximately halfway along its longitudinal length, and a proximal collar flange 54 located in spaced relation to the distal collar flange 56. A circumferential groove 55 is defined between the distal collar flange 56 and the proximal collar flange 54. A central bore or passage 43 extends longitudinally through the inner sleeve 52 and may have a relatively constant cross-sectional profile, or may have a cross-sectional profile that varies along its longitudinal length. The gate opening 42 opens to passage 43. For ease of understanding, it is assumed that the gate opening 42 is disposed on the distal face of the inner sleeve 52 so that the device may be compact, although such is not required in all embodiments as the guard 40 may be located longitudinally elsewhere along the inner sleeve 52. The inner sleeve 52 includes holes 58 for receiving the retaining balls 82, as discussed further below. These holes 58 are advantageously positioned 90° apart at positions approximately 45° off from the flats 44 of gate opening 42. The inner sleeve 52 is designed to move longitudinally (proximally-distally) and rotationally relative to the intermediate sleeve 60.

The intermediate sleeve 60 is generally tubular and includes a bore 62, lateral screw slots 66, and a proximal flange 68. The bore 62 is tapered at least at its upper end, such as at a ramp section 63, so that it narrows in the distal direction. The slots 66 allow screws 80 therethrough while allowing a limited amount of relative longitudinal movement between screws 80 and intermediate sleeve 60. The proximal flange 68 provides seating for one end of spring 88, as discussed below. The lower portion of the bore 62 includes internal threads 64 for mating with corresponding threads 38 on the outside of female driving section 30 proximate the drive recess 32, so that intermediate sleeve 60 may be affixed to female driving section 30.

The intermediate sleeve 60 rests inside a housing referred to as the outer or locking sleeve 70. The outer sleeve 70 is generally tubular with a bore 72 having a shoulder 74 formed therein, and lateral screw holes 76. The spring 88 is disposed between the shoulder 74 and proximal flange 68 of intermediate sleeve 60, thereby biasing the intermediate sleeve 60 proximally relative to outer sleeve 70. The screw holes 76 are threaded and align with screw slots 66, so that screws 80 may be screwed into screw holes 76, through screw slots 66, and into groove 55. This arrangement allows the inner sleeve 52 to move longitudinally a limited amount relative to the outer sleeve 70.

The retaining balls 82 are disposed between the inner sleeve 52 and the intermediate sleeve 60.

The quick-connect assembly 50 may be used to quickly and releasably lock the surgical tool 90 to the drive unit 20. When outer sleeve 70 is moved proximally relative to the female driving section 30, spring 88 is compressed and the action of screws 80 against proximal flange 54 causes inner sleeve 52 to be displaced proximally. This movement results in the retaining balls 82 moving to a wider portion of ramp section 63, thereby allowing the balls 82 to be displaced outwardly. The surgical tool 90 is inserted into the quick-connect assembly 50, with the guard 40 and keying section 110 functioning as described above to prevent insertion of inappropriate surgical tools. As the proximal section 92 of surgical tool 90 is initially inserted, the retaining balls 80 may ride along the bearing surfaces 117, but do not press thereagainst in a clamping fashion. When male driven section 100 is seated in drive recess 32, outer sleeve 70 is released. Spring 88 displaces outer sleeve 70 distally relative to intermediate sleeve 60 and female driving section 30. This causes screws 80 to bear against distal flange 55, pushing inner sleeve 52 distally. The distal displacement of inner sleeve 52 moves balls along ramp section 63 to a narrower section, such that balls 82 are displaced inward. Note that the longitudinal displacement of the inner sleeve 52 may be small, such as one-half to one millimeter, but is sufficient to press the balls 82 inward due to their interaction with ramp section 63. This inward pressing of balls 82 acts to clamp balls 82 against bearing surfaces 117, thereby locking surgical tool 90 against disengagement with female driving section 30, with male driven section 100 seated in drive recess 32. To remove the surgical tool 90, the outer sleeve 70 is again pulled proximally to allow balls 82 to be move outwardly, effectively unlocking the surgical tool 90, and the surgical tool 90 pulled distally to disconnect it from the drive unit 20.

The discussion above regarding the quick-connect assembly 50 has assumed that the outer sleeve 70 is pulled proximally by the surgeon when loading the surgical tool 90. However, such is not required. In some embodiments, the insertion of the surgical tool 90 may cause portions of the surgical tool 90 (e.g., bearing surfaces 117), to push against the balls 82. Due to the orientation of ramp section 63, the balls 82 are allowed to move outward by moving proximally. The push against the balls 82 results in the inner sleeve 52 moving slightly proximally (against bias of spring 88 via outer sleeve 70 and screws 80), such that the relevant portion of the surgical tool 90 is allowed to pass the longitudinal position of the balls 82. Thus, the outer sleeve is not moved proximally relative to the female drive section 30 by the surgeon pulling directly on the outer sleeve 70, but is instead moved proximally indirectly due to the interaction of balls 82 and ramp section 63 in response to insertion of surgical tool 90.

The discussion above has assumed that the drive recess 32 has a double-square cross sectional profile for receiving the square drive profile of the male driven section 100. As indicated above, such is for illustrative convenience, and the drive recess 32 and/or male drive section 100 may have alternative non-round profiles. Merely by way of example, the male drive section 100 may have a square drive profile as shown, with the drive recess having a corresponding “single” square drive profile rather than the double square profile illustrated.

The discussion above has also assumed that balls 82 press clampingly against bearing surfaces 117 formed along corners 116 that are advantageously at the same radial distance from axis 91 as the exterior of base section 93. However, such is not required. For example, in other embodiments, the keying section 110 may include a circumferential groove, and the balls 82 may be pressed inward inside this groove to releasably lock the surgical tool 90 relative to the drive unit 20.

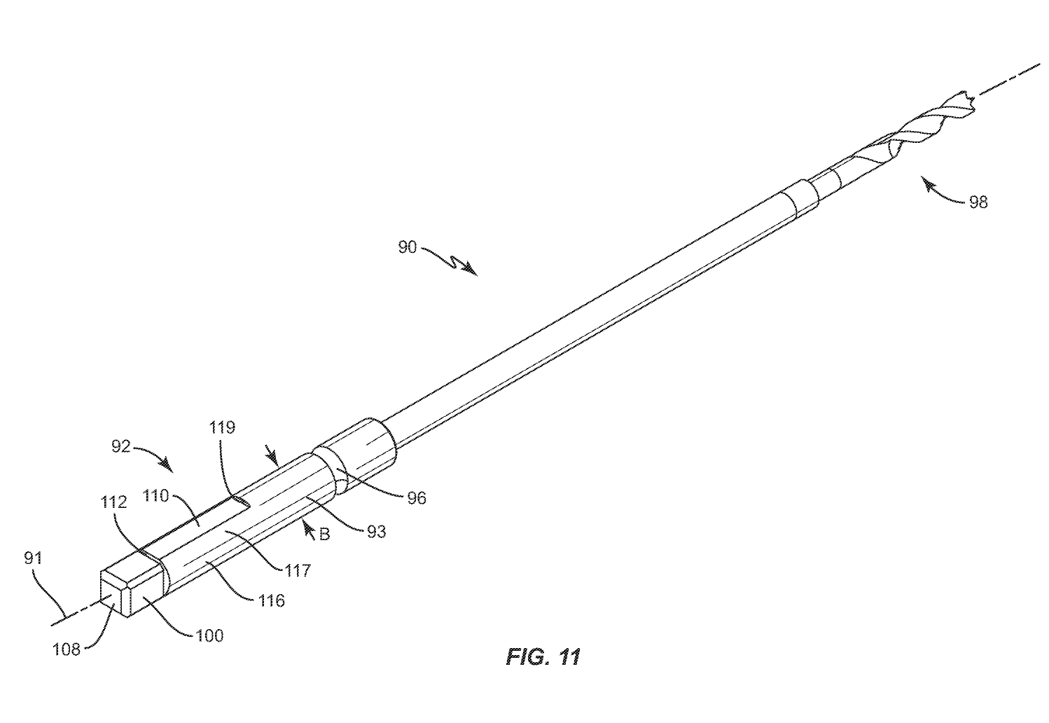

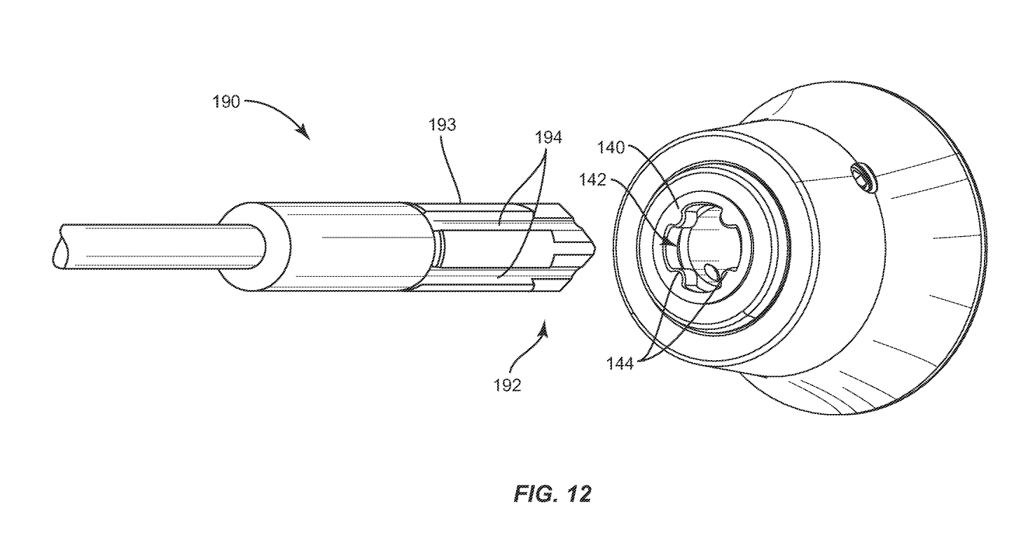

The gate opening 42 and keying section 110 have cross sectional profiles that are selected so that the keying section 110 may pass into the gate opening 42. In the illustrative embodiment, the cross sectional profiles for the gate opening 42 is conceptually a circle with two flat sections, while the cross sectional profile for the keying section is conceptually a circle with four flat sections. Such profiles are believed to provide a strong joint while remaining relatively simple to manufacture. However, as mentioned above, other profiles may alternatively be employed. For example, as shown in FIG. 11, the surgical tool 90 may have two opposing flats 114 separated by two adjoining curving sections 116, rather than four flats 114 separated by four curving sections 116. This configuration of keying section 110 is designed to work with the guard 40 of FIG. 4 in either of two relative orientations. As another example, the alternative guard 140 and alternative surgical tool 190 shown in FIG. 12 employs a plurality of protrusions and grooves to provide the desired blocking of non-conforming surgical tools. In FIG. 12, the proximal section of surgical tool 190 includes a plurality of longitudinally running grooves 194 that extend through the male drive section 192 and the keying section 193. There are four grooves 194 spaced 90° apart, although more or less grooves (symmetric or not) may alternatively be employed. The gate opening 142 of guard 140 includes indentions formed by protrusions 144 that correspond in shape, size, and location to the grooves 194 in surgical tool 90. Note that the presence of the grooves 194 in the male drive section 192 does not adversely affect the acceptance and engagement of male drive section 192 in the drive recess 32, even if the drive recess 32 is unchanged from that discussed above. Of course, the drive recess 32 may alternatively be modified to more closely match the cross sectional profile of the male drive section 192 (e.g., have two or four protrusions added), but such is not believed necessary. Note also that keying section 193 retains its shoulder on its proximal edge that will abut against guard 140 to prevent full insertion of the surgical tool 190 if the gate opening 142 is not of the corresponding configuration.

In some embodiments, the surgical tool 90 may include a circumferential groove 96 disposed distally from the base section 93, for cooperating with attachments to the surgical tool 90.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

The present invention may, of course, be carried out in other specific ways than those herein set forth without departing from the scope of the invention. The present embodiments are, therefore, to be considered in all respects as illustrative and not restrictive, and all changes coming within the meaning and equivalency range of the appended claims are intended to be embraced therein.

What is claimed is:

1. A surgical tool assembly, comprising:
a driving unit comprising:
a rotational power source;
an outer sleeve;
a female driving section coupled to the outer sleeve for rotation about a first rotational axis when receiving rotational input from the rotational power source; the female drive section having a non-circular drive recess therein; the drive recess opening distally and having a maximum cross-sectional dimension, normal to the first axis, of X; the drive recess having a minimum cross-sectional dimension, normal to the first axis, of Z;
a filter mounted to the outer sleeve distally from the drive recess and freely rotatable relative to the outer sleeve; the filter having a filter opening therethrough with a first non-circular cross-section; the filter opening having a minimum cross-sectional dimension, normal to the first axis, of Y; wherein Y is less than X and more than Z;
the drive recess and the filter opening being aligned along the first axis and separated by a first distance.

2. The surgical tool assembly of claim 1 further comprising a surgical tool removably coupled to the driving unit:
the surgical tool comprising:
an elongate drive shaft extending along a longitudinal axis from a proximal section to a distal section;
a male driven section disposed at a proximal end of the drive shaft; the male driven section having a non-circular cross-section normal to the longitudinal axis and a proximal endface;
the male driven section having a maximum cross-sectional dimension, normal to the longitudinal axis, of approximately X;
the male driven section having a minimum cross-sectional dimension, normal to the longitudinal axis, of approximately Z;
a keying feature spaced from the distal endface by a second distance shorter than the first distance; the keying feature having a non-circular cross-section;
the keying feature configured to fit through the filter opening of the filter;
a distal working end;
wherein the female driving section and the male driven section are mated together to rotationally couple the surgical tool to the female driving section;
wherein the filter is rotationally coupled to the female driving section only through the surgical tool;
wherein a distance D between a proximal edge of the keying feature and the proximal endface is shorter than the first distance.

3. The surgical tool assembly of claim 2 wherein the keying feature has a minimum cross sectional dimension K, normal to the longitudinal axis, of more than Y.

4. The surgical tool assembly of claim 2 wherein the cross-section of the keying feature entirely envelopes the cross-section of the male driven section when viewed along the longitudinal axis.

5. The surgical tool assembly of claim 2 wherein:
the cross section of the filter opening of the filter has a first pair of flats spaced Y apart;
the keying feature of the surgical tool has second pair of flats spaced N apart; N being more than Z and less than Y.

6. The surgical tool assembly of claim 5 wherein the surgical tool comprises third pair of flats spaced N apart.

7. The surgical tool assembly of claim 1 wherein the filter comprises a portion of a quick-connect mechanism; wherein the filter is displaceable toward and away from the female driving section; wherein the filter is biased away from the female driving section.

8. The surgical tool assembly of claim 7 wherein the quick-connect mechanism comprises a plurality of balls disposed so as to be urged inward toward the longitudinal axis when the filter is displaced away from the female driving section.

9. The surgical tool assembly of claim 1:
wherein the filter comprises a first plurality of flats;
wherein the drive recess comprises a second plurality of flats;
wherein the second plurality is more numerous than the first plurality.

10. The surgical assembly of claim 1 wherein the rotational power source comprises comprising an electronic drive disposed in the housing and operatively coupled to the female driving section.

11. A surgical tool assembly, comprising:
a driving unit;
a surgical tool removably coupleable to the driving unit; the surgical tool comprising:
an elongate drive shaft extending along a first rotational axis;
a male driven section disposed at a proximal end of the drive shaft; the male driven section having a first non-circular cross-sectional profile normal to the first axis and a proximal endface;
an interference feature disposed distally from the proximal endface of the drive section by a first distance; the interference feature having a first dimension in a radial direction larger than a corresponding portion of the cross-sectional profile of the male drive section;
a distal working end;
the driving unit comprising:
a rotational power source;
a female driving section supported for rotation about a first axis; the female driving section having a distally open drive recess configured to receive the male driven section;
an outer sleeve extending distally from the female driving section and rotationally coupled to the female driving section;
a guard mounted to the outer sleeve distally from the drive recess; the guard having a gate opening therethrough with a second cross-sectional profile that is non-circular; the first and second cross-sectional profiles being different;
the drive recess and the gate opening being aligned along the first axis and separated by a second distance larger than the first distance;
wherein a theoretical cylinder with a diameter of the first dimension cannot pass into the gate opening of the guard;
wherein the assembly is reversibly convertible between a first configuration and a second configuration;
wherein, in the first configuration:
the surgical tool is decoupled from the driving unit;
the guard is freely rotatable relative to the outer sleeve for rotation about the first axis;
wherein, in the second configuration:
the male driven section is matingly received in the drive recess to rotationally couple the surgical tool to the female driving section;
the interference feature extends into the gate opening of the guard;
the working end of the surgical tool is disposed distally from the outer sleeve in spaced relation thereto;
the guard is rotationally coupled to the female driving section through the surgical tool;
wherein the rotational power source, female driving section, guard, and surgical tool are configured such that, in the second configuration, rotational power from the rotational power source is supplied to the surgical tool via the female driving section.

12. The surgical tool assembly of claim 11 wherein the first cross-sectional profile is substantially square.

13. The surgical tool assembly of claim 11 wherein the second cross-sectional profile comprises two diametrically opposed flat sections.

14. The surgical tool assembly of claim 11 wherein the second cross-sectional profile includes a plurality of spaced apart indentions.

15. The surgical tool assembly of claim 14 wherein the surgical tool includes a plurality of groves therein corresponding to the indentions; the grooves extending along the male driven section and distally beyond the interference feature.

16. The surgical tool assembly of claim 11 wherein the guard is mounted to the housing so as to be longitudinally moveable relative to the female drive section; wherein the guard is biased away from the female drive section.

17. A surgical tool assembly, comprising:
a driving unit comprising:
a housing;
a female driving section rotatably coupled to the housing; the female driving section having a drive recess therein having the first cross-sectional profile;
a filter mounted to the housing distally from the recess; the filter having a keyed gate opening therethrough with a second cross-sectional profile that is non-circular; he first and second cross-sectional profiles being different;
the drive recess and the gate opening being aligned along a first axis and separated by a first distance;
wherein the drive recess rotates about the first axis when the female driving section is driven;
a surgical tool removably coupleable to the driving unit; the surgical tool comprising:
an elongate drive shaft extending along a longitudinal axis from a proximal section to a distal section;
the proximal section including a male driven section disposed at a proximal end of the drive shaft and an interference feature disposed distally therefrom;
the male driven section having a third non-circular cross-sectional profile normal to the longitudinal axis and a proximal endface;
the interference feature having a fourth non-circular cross-sectional profile disposed distally from the proximal endface of the drive section by a second distance, with the second distance shorter than the first distance; the interference feature having at least a first dimension in a radial direction larger than any radial dimension of the male driven section; the interference feature sized and configured to fit through the keyed gate opening of the filter;
a distal working end;
wherein, with the male driven section mated to the female driving section:
the second cross-section entirely envelopes the first cross-section when viewed along the longitudinal axis;
the surgical tool rotationally couples the female driving section to the filter;
a most-proximal portion of the interference feature is disposed between the filter and the female driving section.

18. The surgical tool assembly of claim 17 wherein the gate opening of the filter is sized and configured such that a theoretical circle with a diameter of the first dimension cannot fit therewithin.

19. The surgical tool assembly of claim 17 wherein the gate opening of the filter has a cross section with a pair of flats separated by a third distance less than the first dimension.
